(19) United States
(12) Patent Application Publication
(10) Pub. No.: US 2026/0315101 A1
(43) Pub. Date: Oct. 15, 2026

(54) A ROTATIONAL ATHERECTOMY DEVICE

(71) Applicant: CLEARSTREAM TECHNOLOGIES LIMITED, Enniscorthy, County Wexford (IE)

(72) Inventors: Artur SPODOBALSKI, Blackwater (IE); Allan RONAN, Enniscorthy, County Wexford (IE); Eoin CONNAUGHTON, Gorey (IE)

(21) Appl. No.: 19/120,107

(22) PCT Filed: Oct. 12, 2022
(86) PCT No.: PCT/EP2022/078376

§ 371 Date: Apr. 10, 2025

(51) Int. Cl. A61B 17/3207; A61B 17/00; A61B 17/32

(52) U.S. Cl. CPC A61B 17/320758; A61B 17/00234; A61B 2017/00238; A61B 2017/00323; A61B 2017/00398; A61B 2017/00477; A61B 2017/320004

(57) ABSTRACT

A rotational atherectomy device for abrading or cutting tissue. The atherectomy device comprises a flexible outer shaft and a rotatable inner drive shaft, disposed within the outer shaft. The rotational atherectomy device further comprises an abrasive head which is connected to the distal end of the rotatable inner drive shaft through a mechanical joint such that the abrasive head can tilt relative to the rotatable inner drive shaft. The rotational atherectomy device further comprises at least three control wires connected to the distal end of the flexible outer shaft.

### TECHNICAL FIELD

The present disclosure relates to a rotational atherectomy device for abrading or cutting tissue.

### BACKGROUND

Atherosclerosis is a condition where calcified plaque deposits in the walls of a blood vessel lead to narrowing of the blood vessel and increased blood pressure. In severe cases, this can result in peripheral artery disease, coronary artery disease, tissue death or stroke.

Atherosclerosis can be treated in a number of ways such as by angioplasty, where a balloon is used to force the expansion of the blood vessel to allow improved blood flow, a vascular bypass, where a surgical procedure is performed to redirect the blood flow to circumvent the diseased area of the blood vessel, or atherectomy.

Atherectomy is a non-surgical procedure to open blocked blood vessels using a device at the distal end of a catheter to cut or grind atherosclerotic plaque from the walls of the blood vessel. Atherectomy devices can generally be classified into four different types: orbital, rotational, laser and directional.

A rotational atherectomy device uses a rotating abrasive head to cut or grind the atherosclerotic plaque from the walls of the blood vessel. A number of existing rotational atherectomy devices incorporate an abrasive head of fixed diameter. In order to treat larger diameter vessels, the abrasive head is offset towards one side of the vessel wall and the lesion is treated with multiple axial passes, where after each axial pass, the abrasive head is rotated by a set amount. This makes the treatment procedure longer and more complex.

There is hence a need in the art for a new type of rotational atherectomy device which allows controlled and precise movement of the abrasive head to more precisely target specific areas of a vessel wall and therefore reduce the overall treatment time as larger vessels can be treated in fewer axial passes of the abrasive head.

There is further a need in the art for a new type of rotational atherectomy device which minimises the loss of torque between the drive shaft and the abrasive head.

### SUMMARY

In a first aspect of the present disclosure, there is provided a rotational atherectomy device for abrading or cutting tissue. The atherectomy device comprises a flexible outer shaft and a rotatable inner drive shaft, disposed within the outer shaft. The rotational atherectomy device further comprises an abrasive head which is connected to the distal end of the rotatable inner drive shaft through a mechanical joint such that the abrasive head can tilt relative to the rotatable inner drive shaft. The rotational atherectomy device further comprises at least three control wires connected to the distal end of the flexible outer shaft.

In some embodiments, this rotational atherectomy device may allow the abrasive head to be tilted through 360 degrees via longitudinal movement of the control wires. This may allow for controlled and precise movement of the abrasive head to more accurately target specific areas of a vessel wall.

In some embodiments, this rotational atherectomy device may minimise the loss of torque between the drive shaft and the abrasive head.

The mechanical joint may be a universal joint.

In some embodiments, this may minimise the loss of torque between the drive shaft and the abrasive head.

The mechanical joint may be a ball and socket joint.

In some embodiments, this may minimise the loss of torque between the drive shaft and the abrasive head.

The ball and socket joint may comprise first ball positioned within a first socket and a second ball positioned within a second socket. The first ball and socket may be configured to move in a plane orthogonal to the second ball and socket.

The control wires may be connected to the distal end of the flexible outer shaft at regular circumferential intervals.

In some embodiments, this may result in improved control over tilting movement of the abrasive head.

The rotational atherectomy device may comprise at least four control wires connected to the distal end of the flexible outer shaft.

In some embodiments, this may result in improved control over tilting movement of the abrasive head.

The distal ends of the control wires may be connected to the distal end of the flexible outer shaft at regular circumferential intervals.

In some embodiments, this may result in improved control over tilting movement of the abrasive head.

Each of the at least three control wires may be disposed within a respective control wire lumen of the flexible outer shaft.

In some embodiments, this may result in improved control over tilting movement of the abrasive head.

Each of the control wire lumens may terminate in a distal opening disposed proximally of the mechanical joint.

In some embodiments, this may result in improved control over tilting movement of the abrasive head.

The abrasive head may comprise an abrasive surface.

In some embodiments, this may result in more effective removal of plaque from the vessel wall.

The abrasive head may comprise one or more cutting edges.

In some embodiments, this may result in more effective removal of plaque from the vessel wall.

The abrasive head may be substantially spherical shaped.

In some embodiments, this may allow the abrasive head to be used in a forward-facing configuration and a tilted configuration for vessel wall abrasion.

The abrasive head may be substantially cone shaped.

In some embodiments, this may allow a total occlusion of a blood vessel to be more effectively treated, as the force required for tunnelling through the total occlusion may be reduced.

The abrasive head may be substantially ellipsoid shaped.

In some embodiments, this may allow a longer section of the vessel wall to be abraded.

The rotatable inner drive shaft may be flexible.

In some embodiments, this may allow the rotatable drive shaft to better navigate tortuous blood vessel anatomy.

The rotatable inner drive shaft may have a guidewire lumen for accommodating a guidewire.

In some embodiments, this may allow the rotatable atherectomy device to be introduced into a vessel over a guidewire.

The rotational atherectomy device may further comprise a control unit connected to the proximal end of the rotatable inner drive shaft.

The control unit may comprise a motor for providing rotation to the rotatable inner drive shaft.

The control unit may comprise a rotation control mechanism for controlling the speed of rotation of motor.

In some embodiments, this may allow the speed of rotation of the abrasive head to be controlled for more effective removal of plaque from the vessel walls.

The control unit may comprise a tilt control mechanism for controlling the angle and direction of tilt of the abrasive head.

The tilt control mechanism may be connected to the at least three control wires.

In some embodiments, this may provide an operator with improved control of movement of the abrasive head.

The tilt control mechanism may comprise a joystick.

In some embodiments, this may provide an operator with improved control over movement of the abrasive head.

### BRIEF DESCRIPTION OF THE DRAWINGS

To enable better understanding of the present disclosure, and to show how the same may be carried into effect, reference will now be made, by way of example only, to the accompanying drawings, in which:

FIG. 1 schematically shows a rotational atherectomy device with an abrasive head according to one or more embodiments of the present disclosure;

FIG. 2A schematically shows the rotational atherectomy device of FIG. 1 with the abrasive head tilted;

FIG. 2B schematically shows the rotational atherectomy device of FIG. 1 with the abrasive head tilted;

FIG. 3A schematically shows the atherectomy device of FIG. 1 disposed in an artery.

FIG. 3B schematically shows the atherectomy device of FIG. 1 with the abrasive head tilted disposed in an artery.

FIG. 4A schematically shows an embodiment of the abrasive head having a spherical shape.

FIG. 4B schematically shows an embodiment of the abrasive head having a ellipsoidal shape.

FIG. 4C schematically shows an embodiment of the abrasive head having a conical shape;

FIG. 4D schematically shows an embodiment of the abrasive head having a spherical shape;

FIG. 5 schematically shows an alternative embodiment of a rotational atherectomy device according to one or more embodiments the present disclosure.

### DETAILED DESCRIPTION

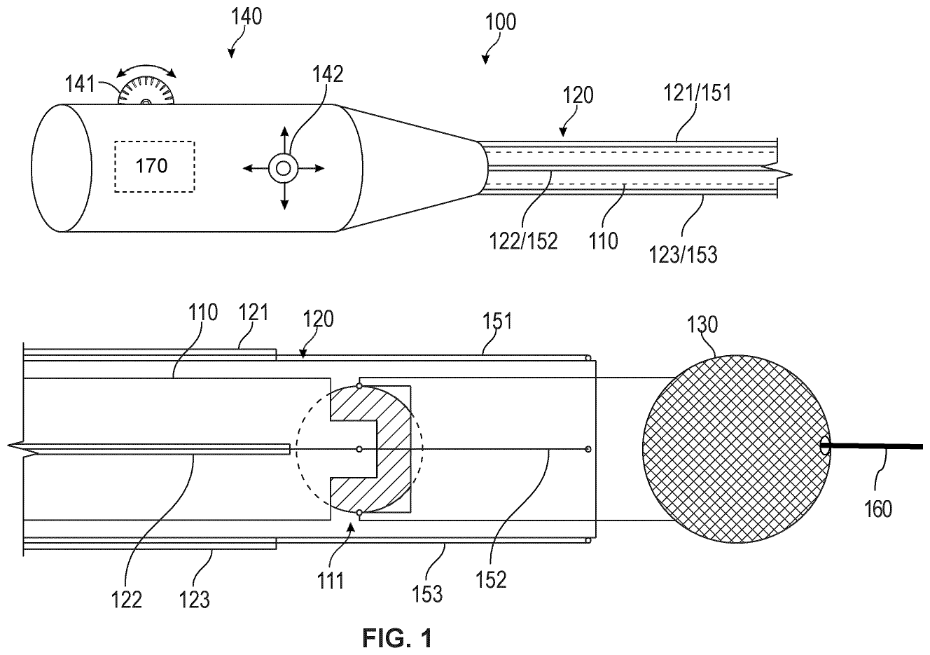

FIG. 1 shows a rotational atherectomy device 100 for cutting or abrading tissue and, specifically, cutting or abrading atherosclerotic plaque from the walls of a blood vessel.

The rotational atherectomy device 100 includes a rotatable inner drive shaft 110 and an abrasive head 130 connected to a distal end of the rotatable inner drive shaft 110 via a mechanical joint so that the abrasive head 130 rotates with the rotatable inner drive shaft 110. In the embodiment of FIG. 1, the mechanical joint is a universal joint 111. The universal joint 111 allows the abrasive head 130 to be tilted relative to the rotatable inner drive shaft 110 so that vessels with larger diameters can be treated with the same rotational atherectomy device 100. The universal joint 111 also provides for efficient torque transfer from the rotatable inner shaft 110 to the abrasive head 130 such that the loss of torque is minimised.

The rotational atherectomy device 100 further comprises a flexible outer shaft 120 which is disposed over the rotatable inner drive shaft 110. Four control wires may be connected to a distal end of the flexible outer shaft 120. FIG. 1 only shows three control wires, a first control wire 151, a second control wire 152 and a third control wire 153. A fourth control wire may be disposed on the other side of the outer shaft 120, opposite the second control wire 152, and is not visible in FIG. 1. The control wires are circumferentially spaced around the outer shaft 120 and may be positioned at regular circumferential intervals. For example, the four control wires may be positioned at 90 degrees intervals circumferentially around the outer shaft 110. The four control wires may extend along the length of the outer shaft 110 and each other control wire may be partially disposed within a respective control wire lumen of the outer shaft 120. For example, the first control wire 151 may be partially disposed within a first control wire lumen 121, the second control wire 152 may be partially disposed within a second control wire lumen 122, the third control wire 153 may be partially disposed within a third control wire lumen 123 and the fourth control wire (not shown) may be partially disposed within a fourth control wire lumen (not shown).

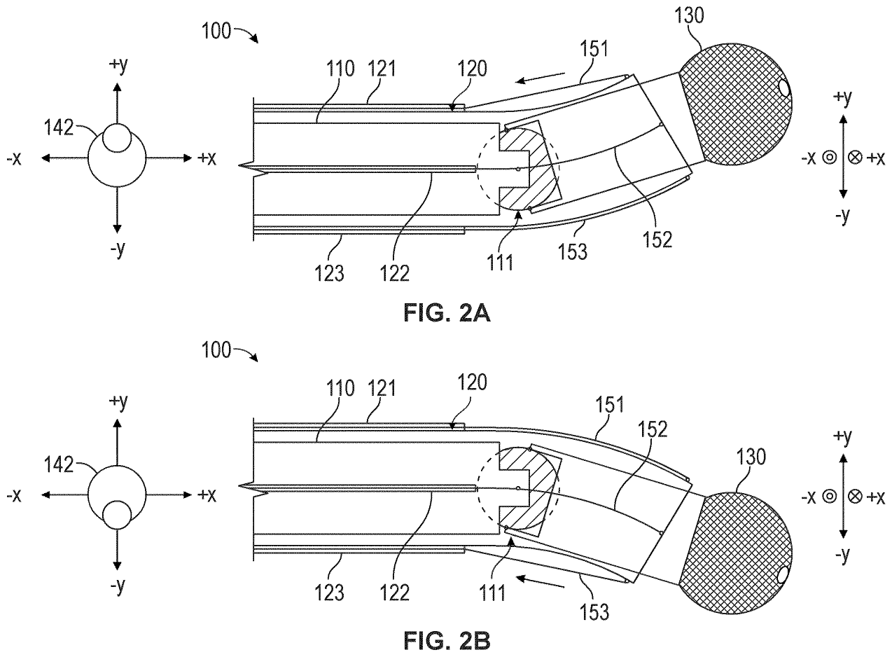

Each of the control wire lumens 121, 122, 123 may terminate in a distal opening which may be disposed proximal of the mechanical joint, such as universal joint 111. A distal portion of the control wires 151, 152, 153 may be exposed and not disposed within a control wire lumen 121, 122, 123. The angle and the amount of tilt of the abrasive head 130 relative to the rotatable control wire 110 may be accurately controlled by pulling one or more of the control wires proximally (see FIG. 2). The rotational atherectomy device 100 may also comprise a guidewire 160 disposed within a guidewire lumen of the rotatable inner shaft 110.

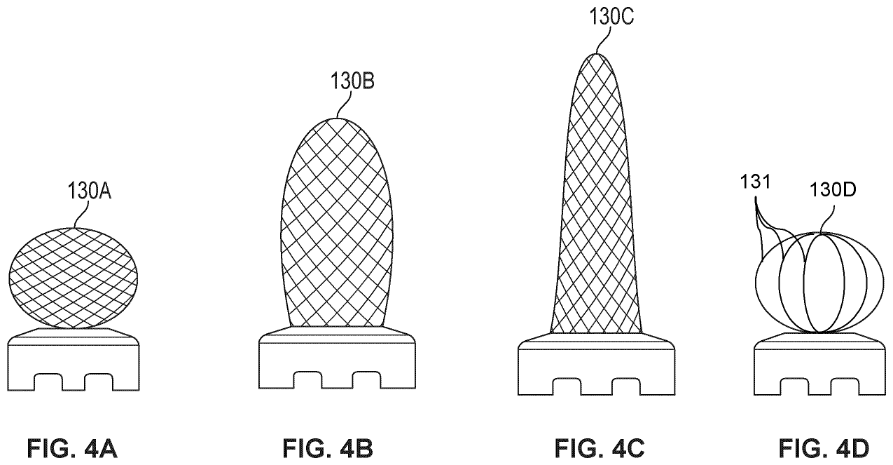

The abrasive head 130 may be substantially spherical and comprise one or more abrasive surfaces. For example, the entire abrasive element 130 may be covered with one abrasive surface or coating. The abrasive surface(s) may be rough to allow it to scrape or abrade plaque from the vessel wall when the abrasive head 130 is rotating. The abrasive head 130 may additionally or alternatively comprise one or more cutting edges (see FIG. 4D). The one or more cutting edges may be sharp edges for cutting plaque from the vessel wall when the abrasive head 130 is rotating. The abrasive head 130 may be made from a number of suitable materials such as, but not limited to, a cobalt base alloy, oxidised zirconium, or tungsten.

The rotational atherectomy device 100 may further comprise a control unit 140 disposed at a proximal end of the rotatable inner shaft 110 and the flexible outer shaft 120. The control unit 140 may be in the form of a handle and comprise a motor 170 which may be connected to the rotatable inner drive shaft 110 for providing rotation to the rotatable inner drive shaft 110 and thereby the abrasive head 130. The control unit 140 may further comprise a rotation control mechanism which may be in the form of rotatable wheel 141 for controlling the speed of rotation of the motor 170 and thereby controlling the speed of rotation of the abrasive head 130. The control unit 140 may further comprise a tilt control mechanism for controlling the angle and direction of tilt of the abrasive head 130. The tilt control mechanism may be in the form of a joystick 142 which is connected to a proximal end of the four control wires. By moving the joystick 142 in a particular direction, one or more of the control wires are pulled in a proximal direction and the direction of tilt of the abrasive head 130 can be determined. The amount that the joystick 142 is moved in that direction then determines the amount of tilt of the abrasive head 130.

The joystick 142 in combination with the four control wires therefore allows the abrasive head 130 to be tilted through 360 degrees and allows for precise control over the direction and amount of tilt of the abrasive head 130.

FIGS. 2A and 2B illustrate how the abrasive head 130 may be tilted relative to the rotatable inner shaft 110. The abrasive head 130 may be tilted in any direction by moving the joystick 142 in a particular direction.

In FIG. 2A, the joystick 142 is moved in the ty direction which causes the first control wire 151 to be pulled proximally. This causes the distal section of the flexible outer shaft 120 to bend resulting in the abrasive head 130 tilting in the ty direction about the universal joint 111.

As shown in FIG. 2B, if the joystick is moved in the −y direction, this causes the third control wire 153 to be pulled proximally. The distal section of the flexible outer shaft 120 bends in the opposite direction resulting in the abrasive head 130 tilting about the universal joint 111 in the opposite −y direction.

Similarly, if the joystick 142 is moved in the +x direction the fourth control wire is pulled proximally and the abrasive head 130 will tilt in the +x direction. If the joystick 142 is moved in the −x direction, then the second control wire 152 is pulled proximally and the abrasive head 130 will tilt in the −x direction.

Movement of the joystick 142 and the abrasive head 130 is not limited to these four directions but can also be moved in any direction between these directions. For example, if the joystick 142 is moved in the ty/+x direction, then both the first control wire 151 and the fourth control wire (not shown) will be pulled proximally, resulting in the abrasive head 130 tilting in the +y/+x direction.

The amount of movement of the joystick 142 in a particular direction will determine the amount of tilt of the abrasive head 130 in that particular direction. The further the joystick 142 is moved in a particular direction, the further the respective control wire or control wires are pulled proximally and the greater the amount of tilt of the abrasive head 130.

The atherectomy device 100 therefore allows the abrasive head to be tilted through 360 degrees in a simple and intuitive manner without rotating the entire device. Furthermore, the atherectomy device 100 provides precise control over the direction and amount of tilt of the abrasive head 130 which allows for very precise abrasion of plaque from a vessel wall.

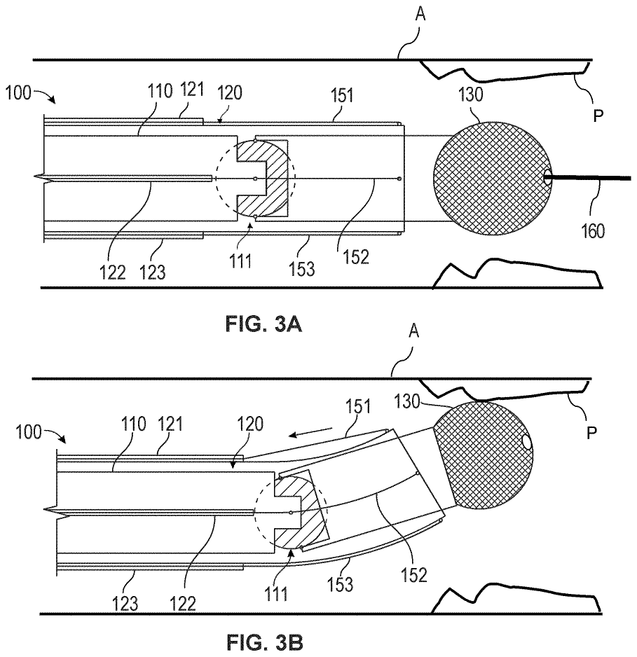

FIGS. 3A and 3B illustrate a method of using the atherectomy device 100 to abrade plaque from the wall of an artery A.

First a suitable access site may be identified. A guidewire 170 may then be introduced into the artery A through the access site and advanced to the treatment site. The treatment site may be, for example, a heavily calcified section of the artery A having significant deposits of plaque P. The artery A may be significantly narrowed or even fully blocked such that blood flow through the artery A is significantly reduced. The rotatable inner shaft 110 with the abrasive head 130 and the outer shaft 120 may then be introduced into the artery A over the guidewire 170 and through the access site.

The atherectomy device 100 may then be advanced over the guidewire 170 until the abrasive head 130 is positioned at the treatment site, as shown in FIG. 3A.

As shown in FIG. 3B, once the abrasive head 130 is positioned at the treatment site, the abrasive head 130 can be tilted to one side by moving the joystick 142 in a particular direction. The rotational motor 170 can be turned on using the rotational control mechanism, for example, by rotating wheel 141 (see FIG. 1) which will cause the rotatable inner shaft 110 and abrasive head to 130 rotate. The rotation will

Cause the abrasive head 130 to cut or scrape the calcified plaque P from the inside of the vessel wall. The speed of rotation of the abrasive head 130 can be controlled by adjusting the amount of rotation of the wheel 141 of the rotation control mechanism.

The abrasive head 130 can then be advanced along the artery wall to remove the plaque P from that side of the artery wall. The joystick 142 can then be moved in a different direction to tilt the abrasive head 130 in a different direction and the operation can be repeated until the entire circumference of the artery A has been treated.

Alternatively, the abrasive head 130 can be advanced while the joystick 142 is rotated which results in the abrasive head 130 tilting through 360 degrees while being advanced. This way the entire circumference of the artery A can be treated in one axial pass which reduces the length of the procedure.

FIGS. 4A-D illustrate different embodiments of the abrasive head 130. FIG. 4A shows a substantially spherical abrasive head 130A. The substantially spherical shape of the abrasive head 130A allows the abrasive head 130A to be used in a forward-facing configuration for treating a total occlusion and a tilted configuration for vessel wall abrasion. The spherical shape of abrasive head 130A also allows the rigid distal section of the rotational atherectomy device 100 to be short to allow the rotational atherectomy device 100 to better manoeuvre tortuous blood vessel anatomy.

FIG. 4B shows a substantially ellipsoidal abrasive head 130B. The substantially ellipsoidal shape of abrasive head 130B allows a longer section of the vessel wall to be abraded when the abrasive head 130B is tilted which may reduce the treatment time.

FIG. 4C shows a substantially conical abrasive head 130C. The substantially conical shape of abrasive head 130C allows the abrasive head 130C to more easily tunnel or drill through a total occlusion by reducing the force required to tunnel or drill through the occlusion.

FIG. 4D shows another embodiment of a substantially spherical abrasive head 130D comprising a number of cutting edges 131 for cutting plaque from the vessel wall.

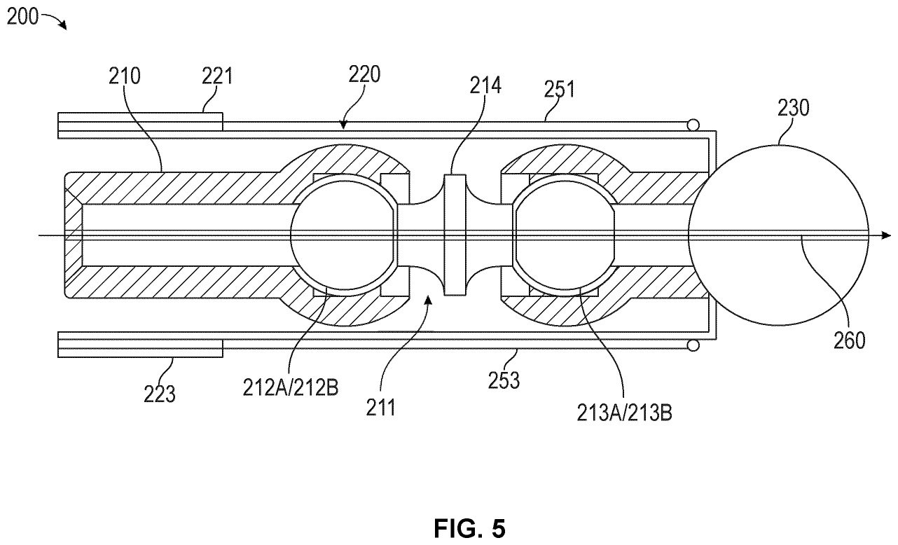

Each of the abrasive heads 130A, 130B, 130C, 130D may be used with the atherectomy devices 100 and 200 (see FIG. 5).

FIG. 5 shows an alternative embodiment of an atherectomy device 200. The atherectomy device 200 is similar to the atherectomy device 100 of FIG. 1 and also comprises a rotatable inner drive shaft 210, a flexible outer shaft 220, an abrasive head 230 and four control wires connected to a distal end of the flexible outer shaft 220. Only two control wires 251 and 253 are shown in FIG. 4. Each of the control wires may be at least partly positioned in a control wire lumen. For example, a first control wire 251 may be partly positioned within a first control wire lumen 221, a second control wire (not shown) may be partly positioned within a second control wire lumen (not shown), a third control wire 253 may be partly positioned within a third control wire lumen 223 and a fourth control wire (not shown) may be partly positioned within a fourth control wire lumen (not shown). The proximal end of the rotatable inner shaft 210 and the flexible outer shaft 110 may be connected to a control unit 140 like that shown in FIG. 1.

The abrasive head 230 is also connected to the rotatable inner shaft 210 with a mechanical joint. However, the mechanical joint is not a universal joint but rather a ball and socket joint 211. The ball and socket joint 211 comprises a first ball 212A positioned within a first socket 212B of the rotatable inner shaft 210. The ball and socket joint 211 further comprises a second ball 213A positioned within a second socket 213B connected to the abrasive head 230. The first ball 212A and the second ball 213A are connected through a connecting element 214, which may be a shaft, for example. The first ball 212A can move within the first socket 212B in a first plane and the second ball 213A can move within the second socket 213B in a second plane which is orthogonal to the first plane. This allows the abrasive head 230 to tilt through 360 degrees relative to the rotatable inner shaft 210. The ball and socket joint 211, similar to the universal joint 111, also minimises the torque lost between the rotatable inner shaft 210 and the abrasive head 230.

A guidewire 260 may be positioned within a guidewire lumen of the rotatable inner drive shaft 210 and may be used to guide the atherectomy device 200 to a treatment site.

The atherectomy device 200 may be used in the same manner as atherectomy device 100 described in relation to FIG. 2A and B above.

Various modifications will be apparent to those skilled in the art.

The mechanical joint is not limited to being a universal joint or a ball and socket joint but may be any suitable mechanical joint which can transfer torque across a bend.

The control wires may not be connected to the distal end of the flexible outer shaft at regular circumferential intervals but may be positioned at irregular circumferential intervals.

The atherectomy device 100, 200 may not have four control wire but may have fewer or more control wires. For example, the atherectomy device 100, 200 may have three, five or six control wires.

The outer shaft 130 may not have any control wire lumens.

The control wire lumens may not terminate proximal to the mechanical joint.

The atherectomy device 100, 200 may not have a guidewire.

The rotatable inner drive shaft 110 may not have a guidewire lumen.

The abrasive head 130 may not comprise an abrasive surface.

The abrasive head 130 may not comprise one or more cutting edges.

The abrasive head 130 may comprise a combination of abrasive surfaces and cutting edges.

The abrasive head 130 is not limited to being spherical, ellipsoid or cone shaped but may take any other suitable shape, for example, cylindrical shaped.

The rotatable inner drive shaft 110 may not be flexible.

The atherectomy device 100, 200 may not comprise a control unit 140.

The control unit 140 may not comprise a motor 170 or a rotation control mechanism for controlling the speed of the motor.

The control unit 140 may not comprise a tilt control mechanism for controlling the angle and direction of tilt of the abrasive head.

The tilt control mechanism is not limited to a joystick but may take any other suitable form, such as a touchscreen, for example.

All of the above are fully within the scope of the present disclosure and are considered to form the basis for alternative embodiments in which one or more combinations of the above described features are applied, without limitation to the specific combination disclosed above.

In light of this, there will be many alternatives which implement the teaching of the present disclosure. It is expected that one skilled in the art will be able to modify and adapt the above disclosure to suit its own circumstances and requirements within the scope of the present disclosure, while retaining some or all technical effects of the same, either disclosed or derivable from the above, in light of his common general knowledge in this art. All such equivalents, modifications or adaptations fall within the scope of the present disclosure.

1. A rotational atherectomy device for abrading or cutting tissue, the atherectomy device comprising:
a flexible outer shaft;
a rotatable inner drive shaft, disposed within the outer shaft;
an abrasive head connected to the distal end of the rotatable inner drive shaft through a mechanical joint such that the abrasive head can tilt relative to the rotatable inner drive shaft;
at least three control wires connected to the distal end of the flexible outer shaft.

2. The rotational atherectomy device of claim 1, wherein the mechanical joint is a universal joint.

3. The rotational atherectomy device of claim 1, wherein the mechanical joint is a ball and socket joint.

4. The rotational atherectomy device of claim 3, wherein the ball and socket joint comprises a first ball positioned within a first socket and a second ball positioned within a second socket, and wherein the first ball and socket are configured to move in a plane orthogonal to the second ball and socket.

5. The rotational atherectomy device of claim 1, wherein the control wires are connected to the distal end of the flexible outer shaft at regular circumferential intervals.

6. The rotational atherectomy device of claim 1, comprising at least four control wire connected to the distal end of the flexible outer shaft.

7. The rotational atherectomy device of claim 6, wherein the distal ends of the control wires are connected to the distal end of the flexible outer shaft at regular circumferential intervals.

8. The rotational atherectomy device of claim 1, wherein each of the at least three control wires is disposed within a respective control wire lumen of the flexible outer shaft.

9. The rotational atherectomy device of claim 8, wherein each of the control wire lumens terminates in a distal opening disposed proximally of the mechanical joint.

10. The rotational atherectomy device of any 1, wherein the abrasive head comprises an abrasive surface.

11. The rotational atherectomy device of claim 1, wherein the abrasive head comprises one or more cutting edges.

12. The rotational atherectomy device of claim 1, wherein the abrasive head is substantially spherical shaped.

13. The rotational atherectomy device of claim 1, wherein the abrasive head is substantially cone shaped.

14. The rotation atherectomy device of claim 1, wherein the abrasive head is substantially ellipsoid shaped.

15. The rotational atherectomy device of claim 1, wherein the rotatable inner drive shaft is flexible.

16. The rotational atherectomy device of claim 1, wherein the rotatable inner drive shaft has a guidewire lumen for accommodating a guidewire.

17. The rotational atherectomy device of claim 1, further comprising a control unit connected to the proximal end of the rotatable inner drive shaft.

18. The rotational atherectomy device of claim 17, wherein the control unit comprising a motor for providing rotation to the rotatable inner drive shaft.

19. The rotational atherectomy device of claim 17, wherein the control unit comprises a rotation control mechanism for controlling the speed of rotation of motor.

20. The rotational atherectomy device of claim 17, wherein the control unit comprises a tilt control mechanism for controlling the angle and direction of tilt of the abrasive head.

21-22. (canceled)
